(19) United States
(12) Patent Application Publication
(10) Pub. No.: US 2025/0041548 A1
(43) Pub. Date: Feb. 6, 2025

(54) SOFT ROBOT INTUBATION DEVICE

(71) Applicant: Vine Medical, Inc., Goleta, CA (US)

(72) Inventors: David HAGGERTY, Goleta, CA (US); James CAZZOLI, Goleta, CA (US); Elliot HAWKES, Goleta, CA (US); David DROVER, Stanford, CA (US); Rohit GIROTRA, San Francisco, CA (US); Changgeng XU, Goleta, CA (US)

(21) Appl. No.: 18/610,070

(22) Filed: Mar. 19, 2024

Related U.S. Application Data
(63) Continuation of application No. PCT/US22/44162, filed on Sep. 20, 2022.
(60) Provisional application No. 63/246,135, filed on Sep. 20, 2021.
(60) Provisional application No. 63/306,805, filed on Feb. 4, 2022.
(60) Provisional application No. 63/399,597, filed on Aug. 19, 2022.

(51) Int. Cl. A61M 16/04; A61B 34/30

(52) U.S. Cl. CPC A61M 16/04; A61B 34/30

(57) ABSTRACT

A soft robot intubation device comprises a compliant body. Upon placement into a bodily orifice and subsequent pressurization therein, the compliant body forms (i) a primary segment extending to the back of the laryngopharynx, (ii) a secondary segment extending from the first segment and into the trachea, and (iii) at least one patency from the first segment to the second segment, providing a pathway from the bodily orifice to the trachea through which ventilation can be provided, either through the patency itself or an endotracheal tube advanced therethrough. The compliant body can comprise walls defining a separate first and second patencies. Pressurization of the first patency forms the primary and secondary segments. Pressurization of the second patency opens the pathway from the bodily orifice to the trachea. Additional elements are provided to secure the device to the anatomy, manipulate the anatomy, or support various device components.

### CROSS-REFERENCE

The present application is a continuation of PCT Patent Application No. PCT/US22/44162, filed Sep. 20, 2022, which claims priority to U.S. Provisional Application No. 63/399,597, filed Aug. 19, 2022, U.S. Provisional Application No. 63/306,805, filed Feb. 4, 2022, and U.S. Provisional Application No. 63/246,135, filed Sep. 20, 2021, each of which is incorporated by reference herein in its entirety.

### TECHNICAL FIELD

The present disclosure relates to medical devices, and more particularly to devices and methods for performing endotracheal intubation.

### BACKGROUND

Tracheal intubation, or intubation, is the placement of a flexible tube into the trachea (windpipe) to maintain an open airway or to serve as a conduit through which to administer certain drugs. It is frequently performed in critically injured, ill, or anesthetized patients to facilitate ventilation of the lungs, including mechanical ventilation, and to reduce the risk of asphyxiation due to an airway obstruction.

The most widely used route is orotracheal, in which an endotracheal tube is passed through the mouth and into the trachea. Intubation is normally facilitated by using a conventional laryngoscope, flexible fiberoptic bronchoscope, or video laryngoscope to identify the vocal cords and pass the tube between them into the trachea instead of into the esophagus. After the trachea has been intubated, a balloon cuff is typically inflated just above the far end of the tube to help secure it in place, to prevent leakage of respiratory gases, and to protect the tracheobronchial tree from receiving undesirable material such as stomach acid.

Due its invasive and uncomfortable nature, intubation is typically performed after administration of general anesthesia, but can also be performed without anesthesia in an emergency. Tracheal intubation is associated with variety complications such as broken teeth or lacerations of the tissues of the upper airway. Potentially fatal complications include pulmonary aspiration of stomach contents which can result in a severe and sometimes fatal chemical aspiration pneumonitis, or unrecognized intubation of the esophagus which can lead to potentially fatal anoxia. Because of these risks, there is an ongoing need for improved devices for intubation that do not suffer from the drawbacks of the current state of the art.

### SUMMARY

Aspects of the present disclosure provide devices for intubation of a subject. An exemplary device may comprise a compliant body configured to form, upon placement into an orifice of a subject and subsequent pressurization therein, (i) a primary segment configured to extend to the back of the laryngopharynx of the subject, (ii) secondary segment configured to extend from the first segment and into a trachea of the subject, and (iii) at least one patency from the first segment to the second segment to provide a pathway from a bodily orifice of the subject to the trachea of the subject. The bodily orifice may be a nostril or a mouth of the subject.

In some embodiments, the compliant body comprises one or more walls defining a first patency and a separate second patency. The first patency permits pressurization of the compliant body to form the primary and secondary segments. The second patency extends from the primary segment into the secondary segment to provide the pathway from the bodily orifice of the subject into the trachea of the subject. The first and second patency may be configured to be pressurized by different pressurization streams. The second patency may be configured to provide ventilation for the subject directly through the pathway provided therefrom. Alternatively, an endotracheal tube may be advanced through the second patency to provide ventilation for the subject.

The device may further comprise an introducer coupled to the compliant body and configured for placement at least partially into the bodily orifice of the subject. The introducer may comprise one or more of a first access port or a second access port, the first access port being open to the first patency and the second access port being open to the second patency. The introducer may comprise a third access port, for example, for cuff inflation.

The portion of the one or more walls defining second patency may be supported by one or more rigid elements. The one or more rigid elements may comprise a plurality of discontinuous rigid elements. The one or more rigid elements may comprise a spring. The one or more rigid elements may comprise at least one continuous, elongate rigid element.

The second patency may be disposed at least partially within the first patency. The first and second patency may be at least partially coaxial.

In some embodiments, the device further comprises a cuff coupled to the secondary segment and configured to protect the at least one patency. The cuff may be at least partially rigid. The cuff may be expandable or inflatable, for example, with a pressure source independent than another pressure source for the primary and secondary segments. The cuff may be coupled to a distal portion of the secondary segment.

In some embodiments, the device further comprises an introducer coupled to the compliant body and configured for placement and securement at least partially into the bodily orifice of the subject. The introducer may be configured to interface with one or more nostrils of the subject. The introducer may be configured to interface with an oropharynx of the subject. The introducer may be configured to interface with a mouth of the subject and comprise a mouthpiece. The device may further comprise a bite block removably coupled to the mouthpiece. The bite block may be configured to at least partially accommodate dentition of the subject.

In some embodiments, the device further comprises a storage compartment coupled to the compliant body and configured to house the compliant body prior to being pressurized. The compliant body may extend at least partially out of storage compartment in response to the pressurization. The device may further comprise an introducer coupled to the compliant body and the storage compartment, the introducer being configured for placement and securement at least partially into the bodily orifice of the subject. The storage compartment may comprise access ports to permit pressurization of the compliant body therethrough, permit ventilation therethrough, and/or cuff inflation.

In some embodiments, the primary segment comprises a first sub-segment and one or more lateral sub-segments, the one or more lateral sub-segments being partially closed to fluid communication with the primary sub-segment.

In some embodiments, the primary segment has a non-circular cross-section such that the primary segment orients to a preferred orientation to the trachea as the primary segment is pressurized. The primary segment may comprise a first sub-segment and one or more lateral sub-segments to provide the non-circular cross-section in combination with the primary sub-segment. The one or more lateral sub-segments may be partially closed to fluid communication with the primary sub-segment. One or more rigid elements may be coupled to a portion of the one or more walls at the first segment to provide the non-circular cross-section.

In some embodiments, the device further comprises at least one shifting element configured to shift the surrounding tissue of the subject and facilitate advancement of the secondary segment into the trachea.

In some embodiments, the device further comprises at least one shifting element configured to shift an epiglottis of the subject and facilitate advancement of the secondary segment into the trachea. The at least one shifting element may comprise at least one lifting element configured to laterally extend from the primary segment as the primary segment is being pressurized. The at least one lifting element may be configured to laterally extend from an anterior side of the primary segment. The at least one lifting element may comprise one or more rigid scales. The at least one lifting element may comprise an inflatable member in fluid communication with the primary segment. The at least one shifting element may comprise a flexible shim configured to slide posteriorly of the epiglottis upon advancement into the laryngopharynx. The flexible shim may be configured to extend from the primary segment in response to pressurization thereof.

In some embodiments, the compliant body is configured to form, upon placement into the bodily orifice and subsequent pressurization therein, a tertiary segment into a first pyriform sinus of the subject to facilitate advancement of the secondary segment into the trachea. The tertiary segment may be configured to extend from the primary segment into the first pyriform sinus of the subject, lifting an epiglottis of the subject to facilitate advancement of the secondary segment into the trachea. The tertiary segment may be configured to extend from the primary segment into the first pyriform sinus of the subject. The secondary segment may be configured to extend from the second segment into the trachea of the subject. The compliant body may be configured to form, upon placement into the mouth and subsequent pressurization therein, a quaternary segment configured to extend into a second pyriform sinus of the subject. The quaternary segment may be configured to extend from the primary segment and into the second pyriform sinus lifting an epiglottis of the subject to facilitate advancement of the secondary segment into the trachea.

In some embodiments, one or more of the primary or secondary segments are configured to evert in response to the pressurization

In some embodiments, the secondary segment has a smaller cross-sectional area than the primary segment.

In some embodiments, the primary segment has a predetermined shape configured to place and orient the secondary segment toward the trachea.

In some embodiments, the primary and secondary segments are unitarily formed.

In some embodiments, the compliant body is made of a thin, flexible material, such as a thin film polymer.

In some embodiments, at least a portion of the compliant body is supported by one or more rigid elements.

Aspects of the present disclosure provide methods for intubation of a subject. In an exemplary method, a compliant body may be placed into a bodily orifice of the subject, the compliant body may be pressurized through at least one patency thereof so that a primary segment of the compliant body everts and extends into the back of a laryngopharynx of the subject and a secondary segment of the compliant body thereafter everts and extends from the primary segment and into a trachea of the subject, and ventilation from the bodily orifice to the trachea may be provided via the pressurized compliant body. The bodily orifice may be a nostril or a mouth of the subject.

In some embodiments, the at least one patency comprises a first patency and a separate second patency. The compliant body may comprise one or more walls defining the first and second patency. Pressurization via the first patency may define the primary and secondary segments. The second patency may provide the ventilation. The second patency may directly provide ventilation for the subject therethrough. The first and second patency may be pressurized by different pressurization streams.

In some embodiments, patency of the second segment is maintained with a cuff coupled to a distal portion of the secondary segment. The cuff may be at least partially rigid. The cuff may be expanded, such as by inflating the cuff, for example, with a pressure source independent than another pressure source for the primary and secondary segments.

In some embodiments, the primary segment may be oriented to a preferred orientation to the trachea as the primary segment is pressurized. Orienting the primary segment may comprise pressurizing a lateral sub-segment of the primary segment extending laterally from a first sub-segment.

In some embodiments, the epiglottis may be shifted to facilitate extension of the secondary segment into the trachea. The epiglottis may be shifted by laterally extending at least one lifting element from the primary segment as the primary segment is being pressurized. The epiglottis may be shifted by inflating at least one lifting element as the primary segment is being pressurized. The epiglottis may be shifted by extending a flexible shim from the primary segment in response to pressurization thereof so that the flexible shim slides posteriorly of the epiglottis.

In some embodiments, pressurizing the compliant body through the at least one patency thereof extends a tertiary segment of the compliant body into at least one pyriform sinus of the subject. The tertiary segment may extend from the primary segment and into the first pyriform sinus of the subject, lifting an epiglottis of the subject to facilitate advancement of the secondary segment into the trachea. The tertiary segment may extend from the primary segment into the first pyriform sinus of the subject, and wherein the secondary segment extends from the primary segment into the trachea of the subject. The compliant body may form, upon placement into the bodily orifice and subsequent pressurization therein, a quaternary segment extending into a second pyriform sinus of the subject. The quaternary segment may extend from the primary segment and into the second pyriform sinus lifting an epiglottis of the subject to facilitate advancement of the secondary segment into the trachea.

In some embodiments, the at least one patency directly provides ventilation for the subject therethrough. Providing ventilation via the at least one patency may comprise advancing an endotracheal tube through the at least one patency and providing ventilation through the endotracheal tube.

In some embodiments, the compliant body is retracted from the trachea and the bodily orifice, such as by applying negative pressure to the compliant body.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings (also “Figure” and “FIG.” herein), of which:

FIGS. 1A-1C depict an embodiment of the intubation device of the present disclosure in progressive states during an intubation procedure.

FIGS. 2A-2B depict an embodiment of the intubation device of the present disclosure having a single-walled or double walled configuration.

FIGS. 3A-3B depict an embodiment of the intubation device of the present disclosure having either an internal or external cuff.

FIGS. 4A-4D an embodiment of the intubation device of the present disclosure having an expandible cuff that is either rigid or inflatable cuff.

FIGS. 5A-5E depict an embodiment of the intubation device of the present disclosure having a rigid element supporting the at least one patency in progressive stages of deployment.

FIG. 6 depicts an embodiment of the intubation device of the present disclosure having an elongate rigid element.

FIGS. 7A-7D depict an embodiment of the intubation device of the present disclosure having a mouthpiece, a storage compartment, a primary segment, a secondary segment, a cuff, a first access port, and a second access port, in progressive stages of deployment.

FIGS. 8A-8D, depict an embodiment of the intubation device of the present disclosure wherein the compliant body of an intubation device has one or more sub-segments.

FIG. 9 depicts an embodiment of the intubation device of the present disclosure wherein the compliant body of the intubation device has one or more rigid elements to provide a non-circular cross-section.

FIGS. 10A-10B depict a device-assisted intubation device having a rigid blade coupled to an intubation device of the present disclosure, according to some embodiments.

FIGS. 11A-11E depict a device-assisted intubation device having a shim coupled to the intubation device of the present disclosure, according to some embodiments.

FIG. 12 depicts a device-assisted intubation device having a stylet coupled to the intubation device of the present disclosure, according to some embodiments.

FIGS. 13A-13G depict midline epiglottis defeat devices a lifting element coupled to a compliant body of the intubation device, according to some embodiments.

FIGS. 14A-14C depict various embodiments of a shim of the intubation device.

FIGS. 15A-15D depict an embodiment of the intubation device of the present disclosure of in progressive states during advancement of a compliant body posterior to a hollow shim.

FIG. 16 depicts an embodiment of the intubation device of the present disclosure having a rigid vertebra coupled to a solid flexible shim.

FIG. 17 depicts asymmetric pyriform exploit epiglottis defeat devices having a tertiary segment configured to enter a first pyriform sinus of a patient, according to some embodiments.

FIGS. 18A-18B depict a symmetric pyriform exploit epiglottis defeat devices having a tertiary segment configured to enter a first pyriform sinus of a patient and a quaternary segment configured to enter a second pyriform sinus of the patient, according to some embodiments.

### DETAILED DESCRIPTION

The following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Generally, present disclosure provides devices and methods for intubating a patient. In some embodiments, the present disclosure provides an intubation device comprising a compliant body that can be placed into a bodily orifice of a patient and pressurized. In some embodiments, pressurization of the compliant body causes a primary segment of the compliant body to extend to the back of the laryngopharynx. In some embodiments, pressurization of the compliant body causes a secondary segment to extend from the primary segment and into the trachea of the patient. In some embodiments, pressurization of the compliant body forms at least one patency from the first segment to the second segment to providing a pathway from a bodily orifice of the patient to the trachea. The compliant body may act as a pathway through which a semi rigid breathing tube (e.g., an endotracheal tube) may be passed to commence artificial breathing. The total length of the compliant body can be predetermined to match different physiology. e.g., by patient size, age, or measured physical characteristics. In some embodiments, the compliant body is disposable, avoiding problems associated with cleaning and reuse of medical devices, such as, for example, hospital acquired infections.

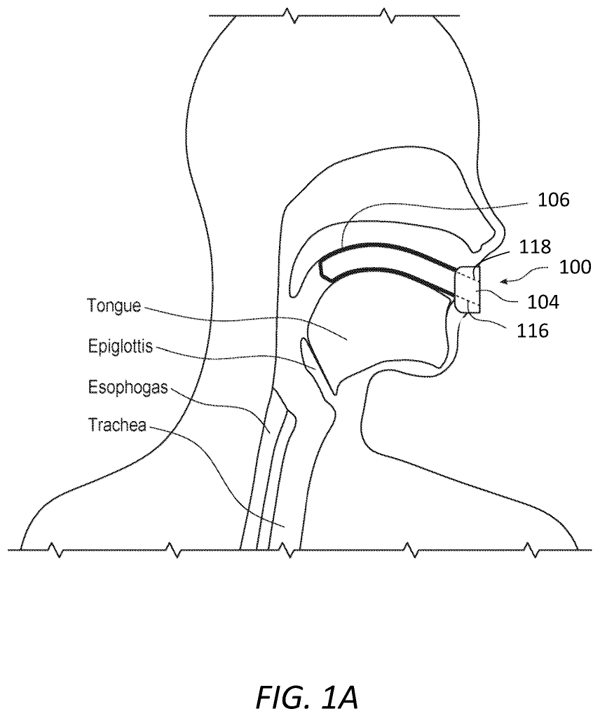

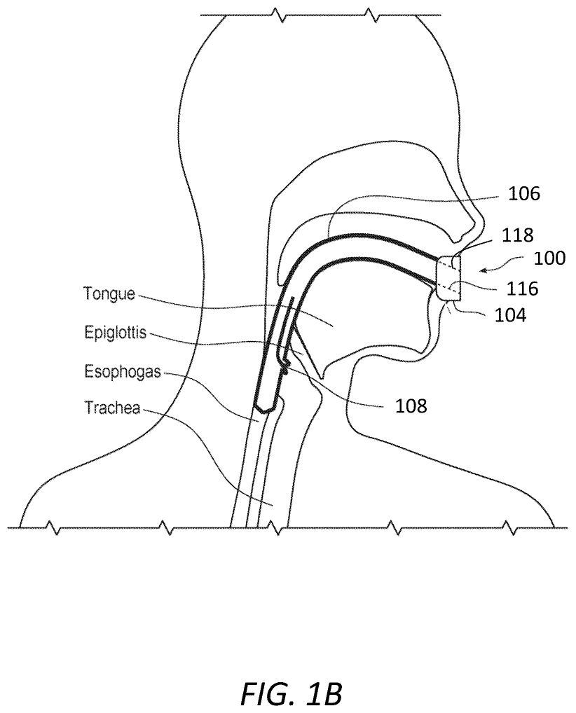

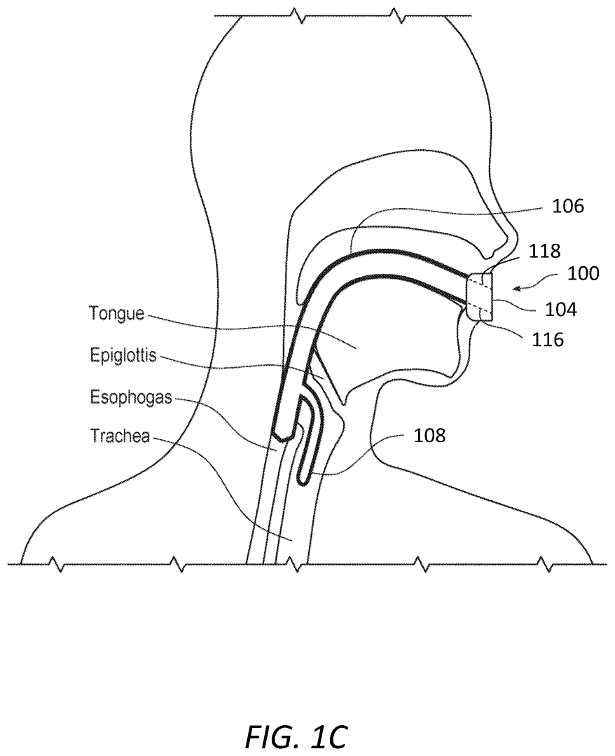

An embodiment of the intubation device 100 is illustrated in FIGS. 1A-1C, in progressive states during an intubation procedure. FIG. 1A shows an intubation device 100 inserted into the mouth of a patient.

In some embodiments, the intubation device 100 comprises a compliant body and configured for placement at least partially into the mouth of the patient. The compliant body may further comprise a primary segment 106 configured to extend to the back if the laryngopharynx of the patient, as shown in FIG. 1B. The compliant body may further comprise a secondary segment 108 configured to extend from the primary segment 106 and into the trachea of the patient, as shown in FIG. 1C. In some embodiments, the secondary segment 108 is a branched segment that is redirected anteriorly causing the secondary segment 108 to extend into the trachea. The compliant body may be formed from biocompatible plastics. In some embodiments, the primary segment 106 and secondary segment 108 are made of polymeric materials. In some embodiments, the polymeric materials are thin-polymeric materials. In some embodiments, at a given pressure, the compliant body has a lower resistance to eversion than to radial stretching such that the body compliant will lengthen upon pressurization instead of ballooning outward. Generally, the diameter and length of both the primary segment 106 and secondary segment 108 can be predetermined according to physiology type or measured physical characteristics of a patient being intubated. e.g., age, gender, according to physical measurements of throat structure, etc. In practice, a practitioner can select an intubation device 100 appropriately sized based upon the physiology of the patient.

In some embodiments, the compliant body has at least one patency from the primary segment 106 to the secondary segment 108. The at least one patency configured to provide a pathway from the mouth to the trachea of the patient.

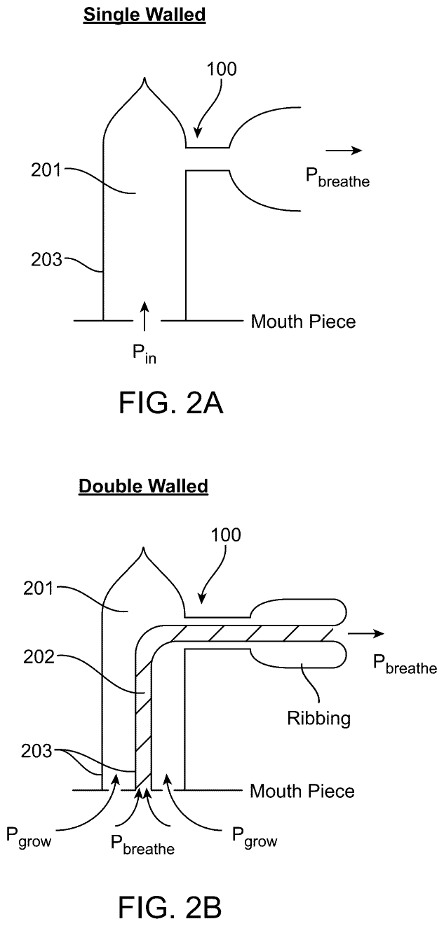

The intubation device 100 may be a single or double-walled device. FIGS. 2A-2B depict the intubation device 100 of the present disclosure having a single-walled or double walled configuration. In some embodiments, the intubation device 100 is a single walled device such that there no fluid (e.g., air or gas) separation. In some embodiments, the intubation device 100 is a double-walled device wherein the compliant body comprises one or more walls 203 defining a first patency 201 and a second separate patency 202. In some embodiments, the first patency 201 and second separate patency 202 provide discrete chambers separating the expansion fluid and ventilation fluid. In some embodiment, separating expansion fluid and ventilation fluid allows the intubation device 100 to employ higher pressures for expansion than would typically be used for ventilation, without subjecting the lungs of the patient to the higher pressures. In some embodiments, the first patency 201 permits pressurization of the compliant body and formation of the primary segment 106 and secondary segment 108. In some embodiments, second separate patency 202 extends from the primary segment 106 into the secondary segment 108 to provide a pathway from the mouth to the trachea of the patient. In some embodiments, the second separate patency 202 is at least partially within the first patency 201. In some embodiments, the first patency 201 and second separate patency 202 are at least partially coaxial.

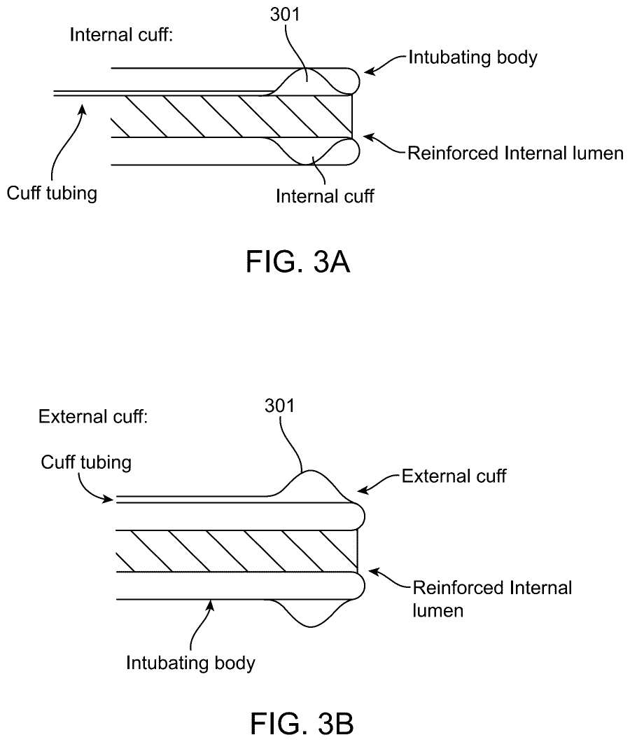

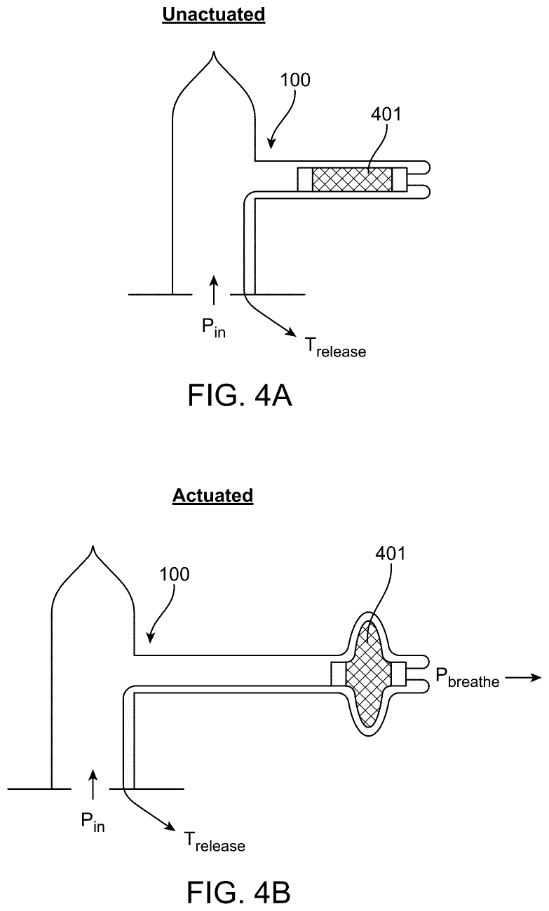

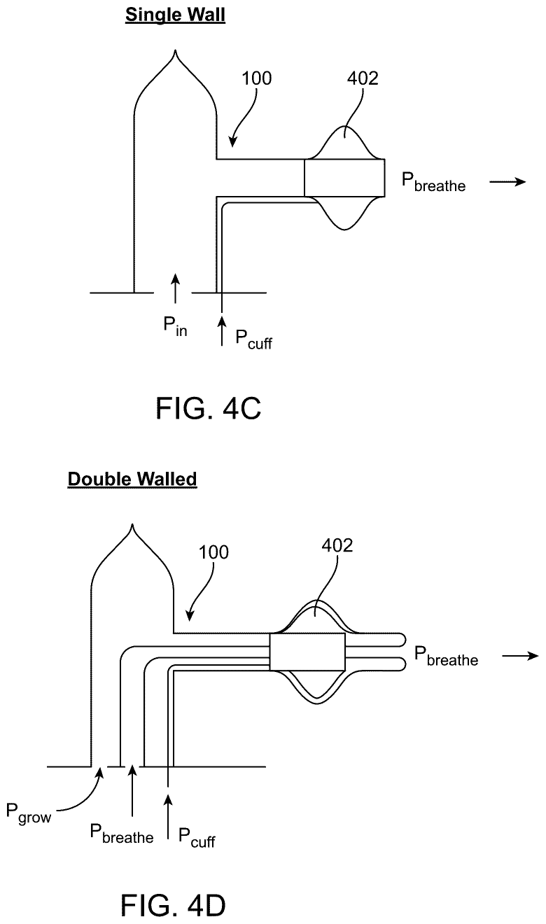

In some embodiments, the intubation device 100 further comprises a cuff coupled to the secondary segment 108, the cuff configured to protect the second separate patency 202. In some embodiments, the cuff is positioned at the distal (i.e., the end farthest from a user of the intubation device 100) end of the secondary segment 108. FIGS. 3A-3B depict an embodiment of the intubation device 100 of the present disclosure having either an internal or external cuff 301. In some embodiments, the cuff is external such this it is affixed to the surface of the secondary segment 108. In some embodiments, the cuff is internal such that disposed within the one or more walls 203 of the compliant body. In some embodiments, the cuff is an expandable element. In some embodiments, the cuff expands radially outward toward the tracheal wall creating a pressure seal (i.e., the expanded volume of the cuff is greater than the tracheal volume). In some embodiments, the cuff is fabricated from biocompatible materials to create an optimal seal with the surrounding tissue. FIGS. 4A-4D depict the intubation device 100 of the present disclosure having an expandible cuff that is either rigid cuff 401 or inflatable cuff 402. In some embodiments, the intubation device 100 comprises a rigid cuff 401. In some embodiments, the rigid cuff 401 is actuated by expansion of the secondary segment 108 via a pass-through connection at the cuff tip and a rigid connection at the cuff base such that eversion of the secondary segment 108 causes compression of the rigid cuff 401 forcing the cuff tip and cuff base together and expansion of the cuff body radially outwards, similar to an arterial stint. In some embodiments, the rigid cuff 401 comprises a locking mechanism to lock the rigid cuff 401 in the actuated state. In some embodiments, the locking mechanism is released by a release wire. In some embodiments, the release wire re-inverts the intubation device 100 until the device is removed from the patient.

In some embodiments, the cuff is inflatable. In some embodiments, the inflatable cuff 402 is inflated using the same pressure source as the primary segment 106 and secondary segment 108. In some embodiments, the distal end of the secondary segment 108 comprises a wider portion such that upon pressurization (i.e., inflation and eversion) the wider portion blocks the trachea. In single-walled intubation devices, positive end-expiratory pressure may be employed to ensure continued isolation from the esophagus. In double-walled intubation devices the expansion and ventilation fluids are separated by the one or more walls 203 such that positive end-expiratory pressure is not needed. In some embodiments, the inflatable cuff 402 is inflated using a pressure source independent from the pressure source for the primary segment 106 and secondary segment 108. In some embodiments, the inflatable cuff 402 is a pneumatic cuff affixed to the distal end of the secondary segment 108. In some embodiments, the pneumatic cuff is inflated using a cuff inflation line similar to the cuff inflation line of an endotracheal tube. In some embodiments, the pneumatic cuff is actuated by soft valves manufactured into the intubation device 100. In some embodiments, the secondary segment 108 is reinforced to protect the at least one patency (i.e., prevent collapse of the at least one patency) from the mouth to the trachea of the patient. In some embodiments, the secondary segment 108 is reinforced at the position of the cuff.

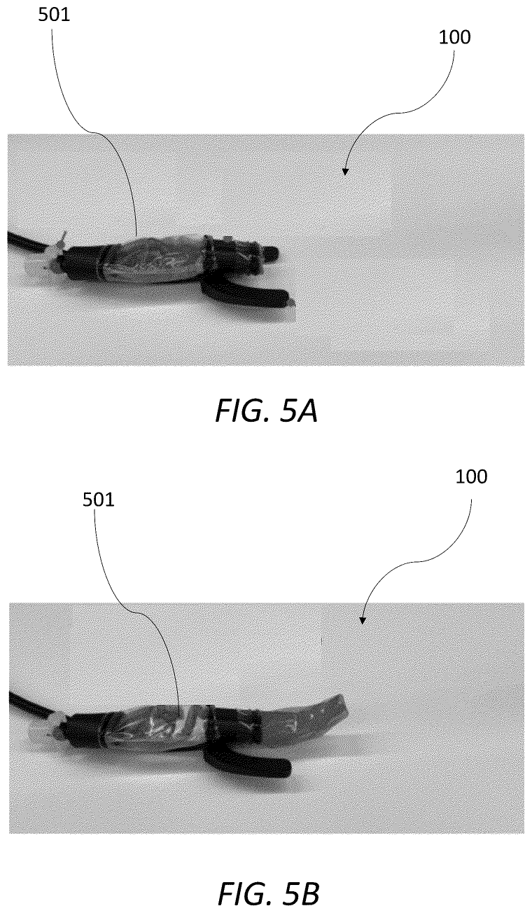

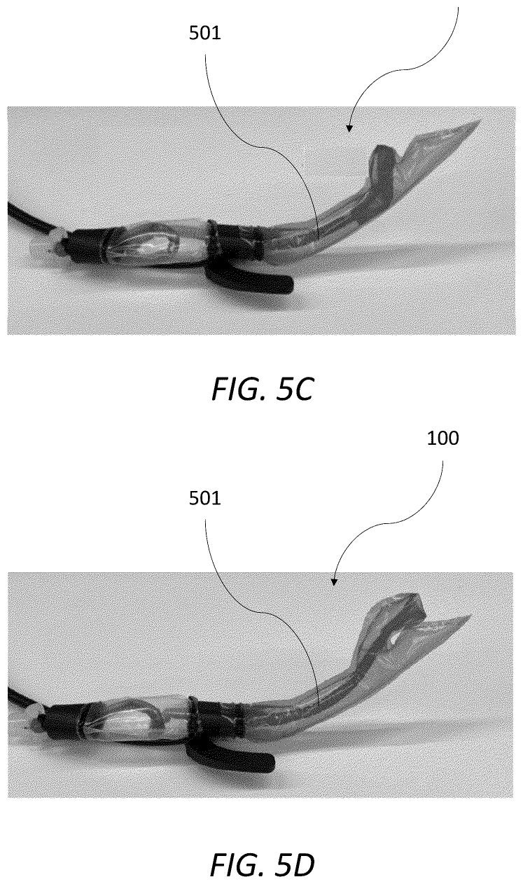

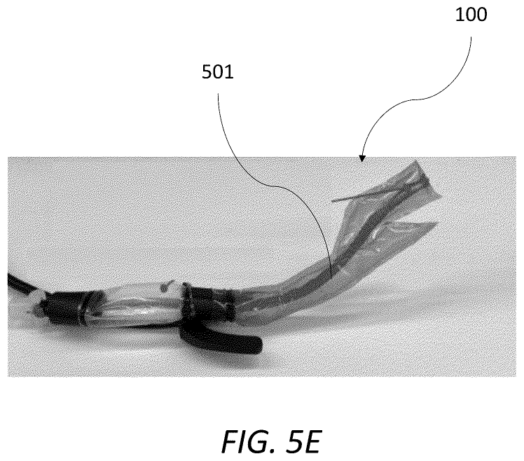

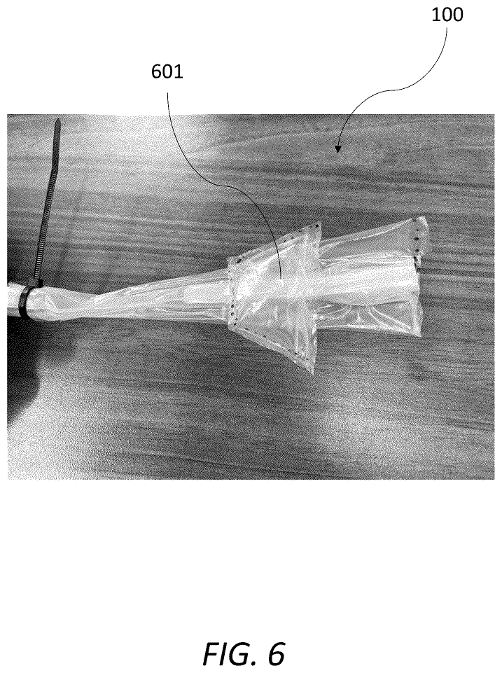

FIGS. 5A-5E depict an embodiment of the intubation device 100 of the present disclosure having a rigid element 501 supporting the at least one patency in progressive stages of deployment. In some embodiments, the one portion of the one or more walls 203 defining the second separate patency 202 is supported by one or more rigid elements. In some embodiments, the one or more rigid elements comprises a plurality of discontinuous rigid elements. In some embodiments, the plurality of discontinuous rigid elements comprises an assembly of small sections of tubing appropriately spaced. In some embodiments, the one or more rigid elements comprise a spring. In some embodiments, a thin-film, such as polymer, is wrapped or pressure fitted around the spring. In some embodiments, the one or more rigid elements comprises at least one continuous, elongate rigid element. FIG. 6 depicts an embodiment of the intubation device 100 of the present disclosure having an elongate rigid element 601. In some embodiments, rigid element provides better staging for deployment such that rigid element sequences expansion of the compliant body. In some embodiments, the elongate rigid element 601 allows passage of a rigid ventilation tube, such as an endotracheal tube through the at least one patency. In some embodiments, a rigid ventilation tube is affixed to the distal end of the secondary segment 108, the inflation acts as a pulling force as the secondary segment 108 everts, allowing for the automatic delivery of a rigid ventilation tube. In some embodiments, a biologically compatible self-hardening material infused into the compliant body of intubation device 100, which permits hardening the intubation device 100 inside the body to produce its own rigid ventilation tube.

In some embodiments, the intubation device 100 further comprises an introducer 104 removably coupled to the compliant body such that after deployment of the compliant body the introducer 104 may be separated from the compliant body. In some embodiments, the introducer 104 comprises one or more of a first access port 116 or a second access port 118, the first access port 116 being open to the first patency 201 in the compliant body and the second access port 118 being open to the second patency 202 in the compliant body. The one or more of a first access port 116 or a second access port 118 configured to permit fluid/pressure transfer into the compliant body of the intubation device 100 to evert and expand the compliant body. In some embodiments, the first patency 201 and the second patency 202 may be pressurized by different pressure streams or devices. In some embodiments, the second patency 202 is configured to provide ventilation to the patient through the pathway created therethrough. In some embodiments, the introducer 104 includes a passage through which the semi-rigid breathing tube can be passed down to the trachea through the second separate patency 202. Couplings to the introducer 104 for inflation are compatible with current medical technology. e.g. a Luer lock.

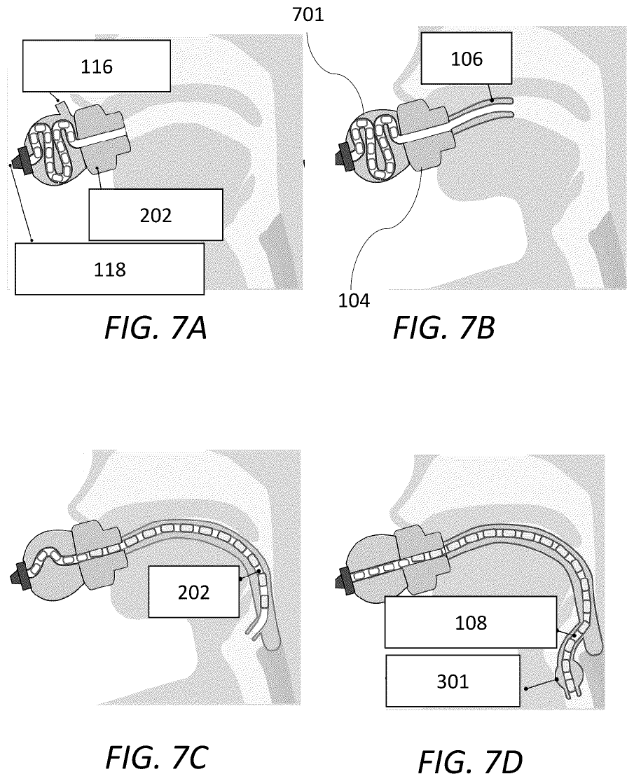

FIGS. 7A-7D depict an embodiment of the intubation device 100 of the present disclosure having a mouthpiece, a storage compartment 701, a primary segment 106, a secondary segment 108, a cuff, a first access port 116, and a second access port 118 in progressive stages of deployment. In some embodiments, the introducer 104 is a mouthpiece. In some embodiments, the mouthpiece is formed from a biocompatible plastic or firm rubber compound. In some embodiments, the mouthpiece has the general anatomy of the human face and mouth. In some embodiments, the mouthpiece is employed either externally or internally to the mouth of the patient. The mouthpiece may further include a protrusion to depress the tongue and a bite block which may be removably coupled to the mouthpiece such that the mouthpiece may be removed upon deployment of the compliant body. In some embodiments, the bite block comprises a hollow body allowing passage of the compliant body therethrough, as shown in FIGS. 7A-7D. The hollow bite block allows the compliant body to be stored behind anteriorly. In some embodiments, the bite block has a recess into which the teeth can fall to hold the intubation device 100 inside the mouth and set a standard “zero” point reference from which extension of the intubation device 100 can be determined. In some embodiments, the bite block is oblately shaped, such that, when the compliant body is deployed, the bite block rests between the patient's molars to protect the compliant body from kinking due to biting. Preferred materials for the mouthpiece include medical grade silicone, polyurethane, or polyethylene. The mouthpiece may be sized according to anatomical characteristics. The mouthpiece may house mechanical components that allow for actuation (e.g. buttons) and an indicator to show whether or not it has been used. In some embodiments, the mouthpiece can connect to and/or include a pressure reservoir, an actuation mechanism to commence intubation, and mechanical and electrical elements to actuate and control the intubation device 100.

In some embodiments, the intubation device 100 further comprises a storage compartment 701 coupled to the compliant body and configured to house the compliant body prior to being pressurized. In some embodiments, the compliant body extends at least partially out of storage compartment 701 in response to the pressurization. In some embodiments, the introducer 104 is coupled to the storage compartment 701. In some embodiments, the storage compartment 701 comprises an access port to permit pressurization of the compliant body. In some embodiments, the storage compartment 701 may be transparent to allow the user to visualize when the reinforced segment is fully deployed.

In some embodiments, the cross-sectional area of the primary segment 106 is such that when pressurized the primary segment 106 fills the oral cavity and oropharynx, lifting the lower jaw and protruding it forward and down. Fluid (e.g., air or a gas) is delivered through the first access port 116 with sufficient pressure to slowly inflate and invert the primary segment 106 until it reaches the back of the laryngopharynx. Once the primary segment 106 reaches the laryngopharynx, the secondary segment 108 may be actuated or pressurized. The primary segment 106 can be shaped in a pre-determined non-linear shape (when extended) and have additional features to introduce specific pressure points to protrude the mandible, lift the epiglottis, and expose the trachea. Methods for shaping and producing firmness of the material in particular sections may be found in US Patent Publication US2019/0217908 to Hawkes et al . . . which is incorporated herein by reference.

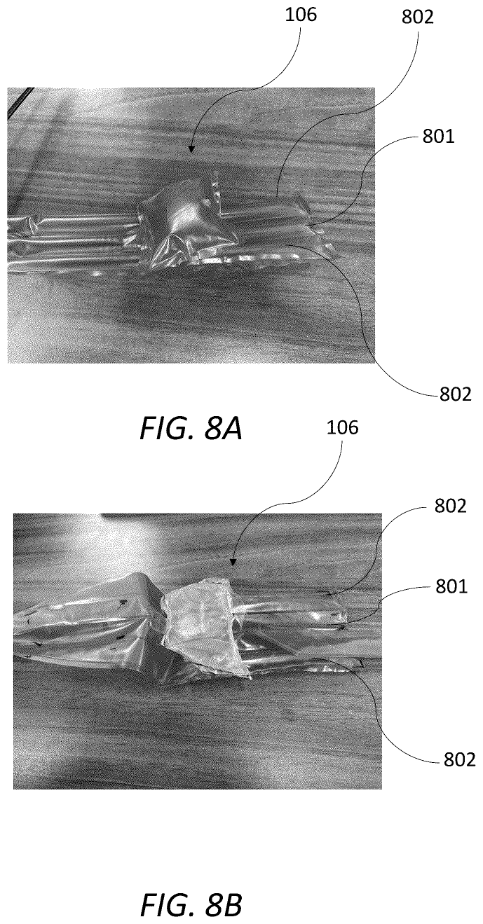

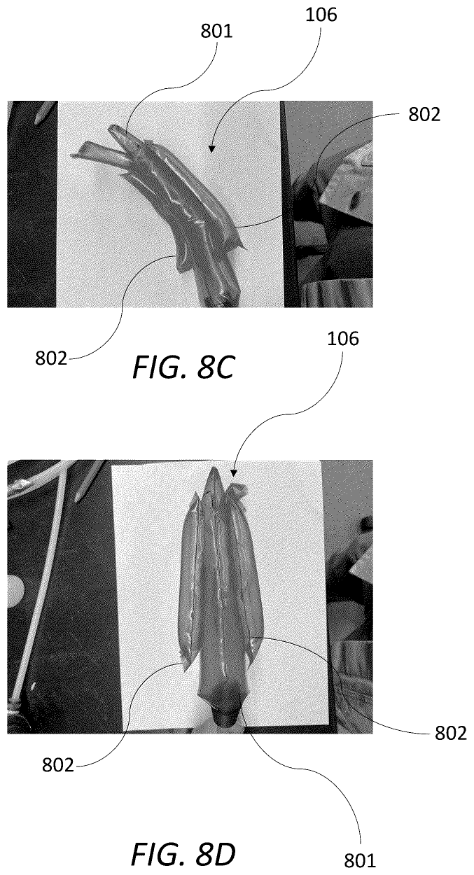

FIGS. 8A-8D, depict a compliant body of an intubation device 100 having one or more sub-segments. In some embodiments, the intubation device 100 may comprise one or more sub-segments configured to provide the compliant body with a non-circular cross-section or to center the primary segment 106 to the anatomy of the patient. In some embodiments, the non-circular cross section is oblate. In some embodiments, the primary segment 106 comprises a first sub-segment 801 and one or more lateral sub-segments 802 configured to provide a non-circular cross-section in combination with the first sub-segment 801, as shown in FIGS. 8A-8B. In some embodiments, the one or more sub-segments configure to provide a non-circular cross-section prevent wrinkling and rotation and yaw of the primary segment 106. In some embodiments, the primary segment 106 comprises a first sub-segment 801 and one or more lateral sub-segments 802 configured to center primary segment 106, as shown in FIGS. 8C-8D. In some embodiments, the one or more sub-segments configured to center the primary segment 106 have a cross-sectional width larger than the width of the anatomy of the patient, such that the upon pressurization the primary segment 106 distributes the fluid (i.e., air or gas) volume between the one or more sub-segments to achieve the lowest energy (i.e., centered to the anatomy of the patient). In some embodiments, the one or more lateral sub-segments 802 being partially closed to fluid communication with the primary sub-segment 801. In some embodiments, the one or more sub-segments may comprise 1, 2, 3, 4, 5, 6, 7, 8, 9, 10, or more sub-segments, or however many are necessary to achieve the desired effect.

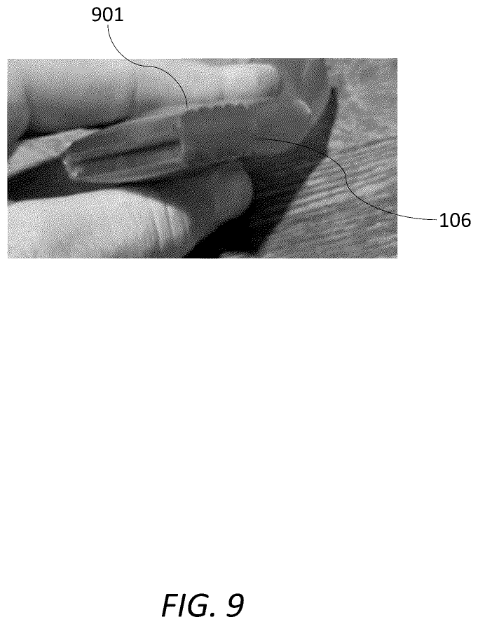

FIG. 9 depicts a compliant body of the intubation device 100 having one or more rigid elements 901 to provide a non-circular cross-section. In some embodiments, the intubation device 100 may comprise one or more rigid elements 901 coupled to a portion of the one or more walls 203 at the primary segment 106 to provide a non-circular cross-section. In some embodiments, the one or more rigid elements configure to provide a non-circular cross-section prevent wrinkling and rotation of the primary segment 106. As shown in FIG. 9, the one or more rigid elements enable the primary segment 106 to have a wide and flat cross-section. In some embodiments, the one or more rigid elements may be positioned symmetrical such that the one or more rigid elements are positioned oppositely on the one or more walls 203 of the primary segment 106. In some embodiments, the one or more rigid elements may be asymmetric. In some embodiments, one or more rigid elements 901 permit inversion and eversion of the compliant body.

In some embodiments, the intubation device 100 that expands to the trachea beyond the vestibular folds. In some embodiments, the distal end of the compliant body 102 terminates with at least one patency or a temporarily sealed distal tip that can be breached or penetrated, such that after pressurization of the compliant body 102 (i.e., eversion), the compliant body 102 provides at least one patency configured to provide a pathway from the mouth to the trachea of the patient. In some embodiments, the distal tip comprises a perforated seal. In some embodiments, the distal tip is elastic. In some embodiments, the perforated or elastic distal tip may be breached using a breathing tube. In some embodiments, the perforated or elastic distal tip provides feedback to a practitioner when the breathing tube breaches the distal tip of the compliant body 102.

In some embodiments, the intubation device 100 is intended to be introduced through a nasal orifice. In some embodiments, the intubation device 100 is introduced through either or both nares. In some embodiments, the introducer 104 directs the primary segment 106 inferiorly between the hard palate and the inferior turbinate. In some embodiments, the primary segment 106 comprises a shifting element. In some embodiments, the primary segment 106 comprises a shifting element for lifting the epiglottis to allow expansion of the primary segment 106 into the laryngopharynx to access the trachea therethrough. In some embodiments, the primary body 106 has a predetermined shape configured to place and orient the secondary segment 108 toward the trachea.

In some embodiments, the compliant body of the intubation device 100 is inverted from the distal tip proximally (i.e., the end closest to a user of the intubation device 100), to be stored in the storage compartment 701 of the intubation device 100. In some embodiments, the intubation device 100 is sterilized and packaged for single use, and packaging and device may include markings to ensure proper use by practitioners.

In some embodiments, the intubation devices are device-assisted intubation devices which require a medical provider to manipulate the tissue of the airway to allow passage of the intubation device. For example, a traditional laryngoscope coupled to the intubation devices may be used to lift the epiglottis allowing passage of the intubation device. In some embodiments, the intubation devices are midline epiglottis defeat devices which autonomously manipulate the tissue of the airway to allow passage of the intubation device. In some embodiments, the intubation devices are pyriform exploit epiglottis defeat devices that exploit the fact that the human body retains a natural opening in the airway even when the body is sedated or in a supine position. For example, the pyriform fossa is a natural opening through which air can pass while the surrounding tissue is otherwise collapsed on the pharyngeal wall. The pyriform exploit epiglottis defeat devices are designed to pass around the tissue of the airway, such as, for example, the epiglottis.

### Device Assisted Intubation Devices

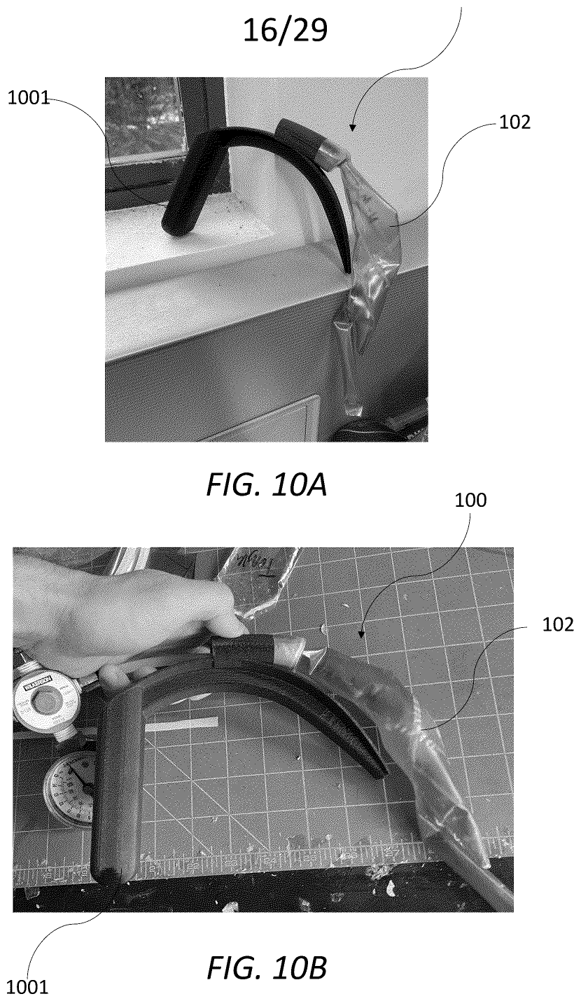

The intubation devices of the present disclosure may be device-assisted intubation devices. In some embodiments, the device-assisted intubation devices require manual manipulation of the tissue of the airway to allow passage of the intubation device. In some embodiments, the device-assisted intubation devices are epiglottis lifting devices. FIGS. 10A-10B depict a device-assisted intubation device comprising a rigid blade 1001 coupled to the intubation device 100. In some embodiments, the intubation device 100 is attached to the superior side of a rigid blade 1001, such as a laryngoscope blade, designed to lift the soft palate from the vallecula, as in a traditional laryngoscopy. After using the rigid blade 1001 to manipulate the airway tissue, a medical provider can pressurize the compliant body 102 of the intubation device 100 such that the compliant body 102 extends posteriorly of the rigid blade 1001 past the epiglottis. In some embodiments, the compliant body 102 further comprises an endotracheal tube (not shown) configured to extend from the compliant body 102 upon pressurization of the compliant body 102. In some embodiments, the intubation device 100 is detached from the rigid blade 1001 to allow the rigid blade 1001 to be removed.

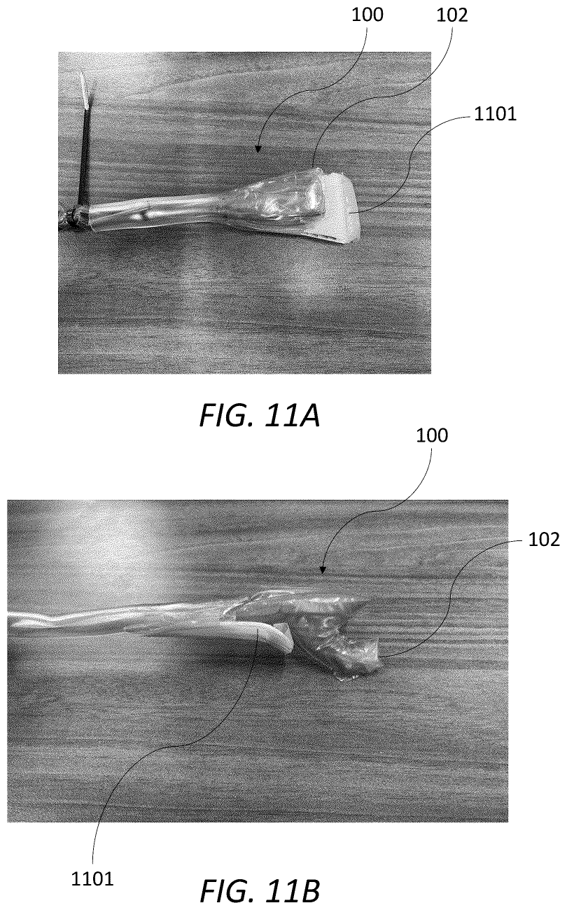

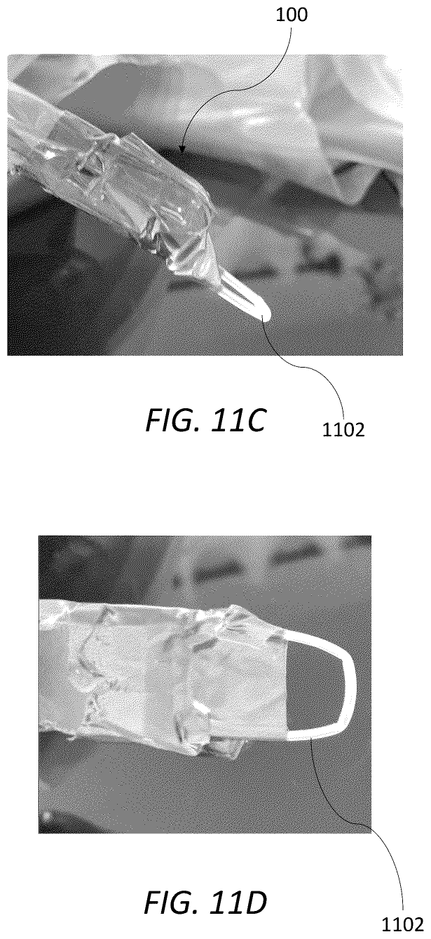

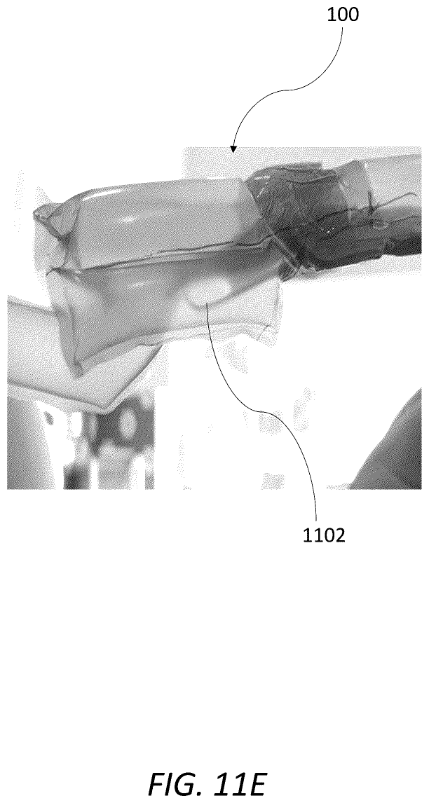

In some embodiments, the device assisted intubation devices are epiglottis shimming devices. In some embodiments, the flexible shim has a high aspect ratio. In some embodiments, the flexible shim has an aspect ratio of 10:1 or higher. In some embodiments, the flexible shim has low stiffness. In some embodiments, the flexible shim is a low friction element allowing the shim to maneuver without encountering significant resistance by the surrounding tissue. In some embodiments, the flexible shim is affixed to the superior side of a rigid blade, such as a laryngoscope blade, and the flexible shim is manually advanced into the hypopharynx. FIGS. 11A-11E depict a shim coupled to the intubation device 100. In some embodiments, the shim is a solid flexible shim 1101, as shown in FIGS. 11A-11B. In some embodiments, the shim is an encased extruded metal wire, or hollow shim 1102, as shown in FIGS. 11C-11E. In some embodiments, the hollow shim 1102 reduces friction, and allows the secondary segment 108 of the compliant body 102 to deploy within the hollow section of the hollow shim 1102 to increase reliability across anatomies.

In some embodiments, hollow shim 1102 or solid flexible shim 1101 slips posterior to the epiglottis. Once past the epiglottis, an intubation device 100 affixed to the superior side of the hollow shim 1102 or solid flexible shim 1101 is pressurized causing the compliant body to extend posterior to the solid flexible shim 1101, thereby passing the epiglottis without inversion (or lifting). FIG. 11E depicts the compliant body of the intubation device 100 deployed posterior to the hollow shim 1102. In some embodiments, the compliant body further comprises an endotracheal tube (not shown) configured to extend from the compliant body upon pressurization of the compliant body. In some embodiments, the intubation device 100 is detached from the solid flexible shim 1101 or hollow shim 1102 allowing the solid flexible shim 1101 or hollow shim 1102 to be removed.

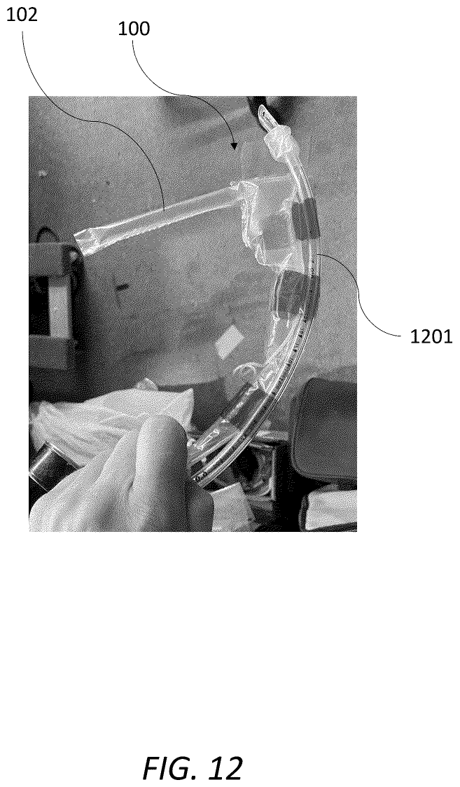

Other embodiments of device-assisted intubation devices include stylets 1201 having the intubation device 100 attached, as shown in FIG. 12. In some embodiments, the semi-rigid stylet 1201 is advanced near the laryngeal inlet, defeating the epiglottis. After epiglottis defeat, the compliant body 102 is pressurized. In some embodiments, the compliant body 102 further comprises an endotracheal tube (not shown) configured to extend from the compliant body 102 upon pressurization of the compliant body 102.

The device-assisted intubation devices may further include any of the features of any embodiment of the present disclosure, for example, embodiments of the device-assisted intubation devices may be combined with embodiments of the midline epiglottis defeat devices and pyriform exploit epiglottis defeat devices.

### Midline Epiglottis Defeat Devices

The intubation devices of the present disclosure may be midline epiglottis defeat intubation devices. In some embodiments, the midline epiglottis defeat devices autonomously manipulate the tissue of the airway to allow passage of the intubation device. In some embodiments, the midline epiglottis defeat devices are epiglottis lifting devices. In some embodiments, the intubation device 100 further comprises at least one shifting element configured to manipulate (e.g. shift, lift, compress, re-orient) a portion of the anatomy of the patient. In some embodiments, the intubation device 100 further comprises at least one shifting element configured to shift an epiglottis of the patient and facilitate advancement of the secondary segment 108 into the trachea. In some embodiments, the intubation device 100 further comprises at least one shifting element configured to shift the tongue to allow advancement of the primary segment 106. In some embodiments, the at least one shifting element is affixed on the lateral side of the primary segment 106. In some embodiments, the at least one shifting element comprises at least one lifting element configured to laterally extend from the primary segment 106 as the primary segment 106 is being pressurized. In some embodiments, the at least one lifting element is configured to laterally extend from an anterior or superior side of the primary segment 106.

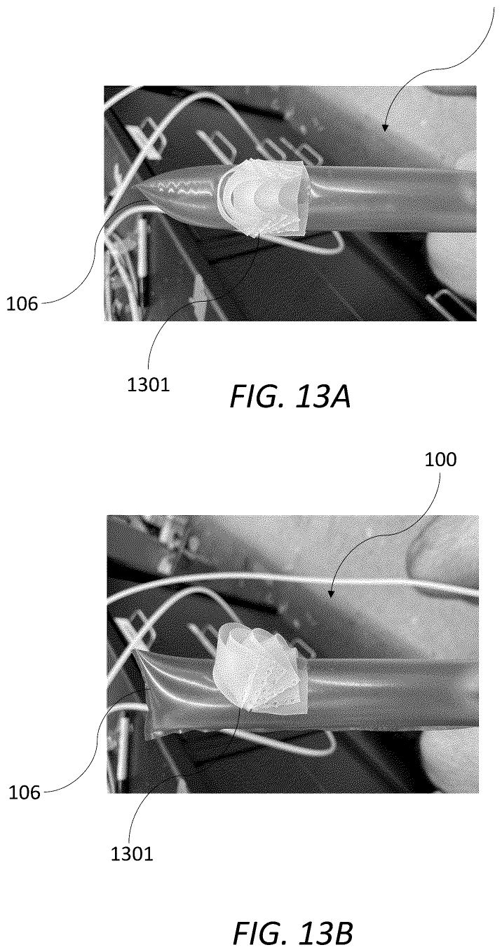

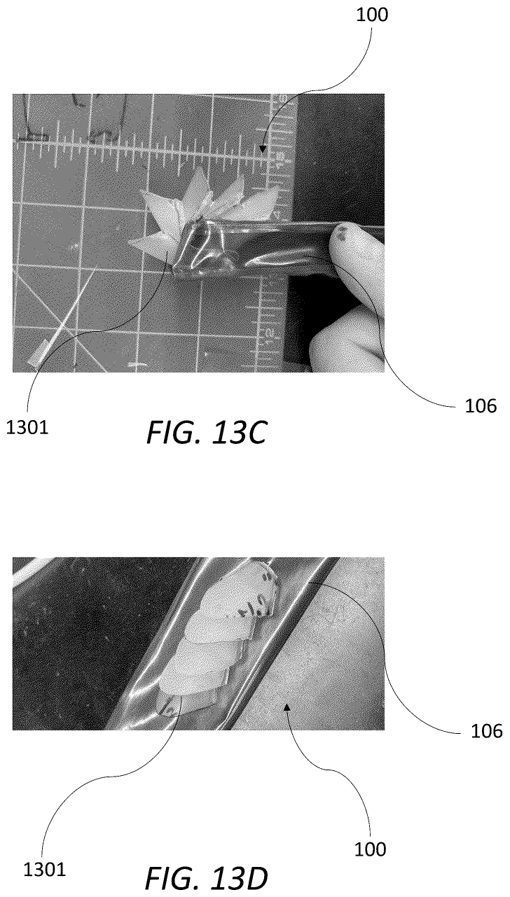

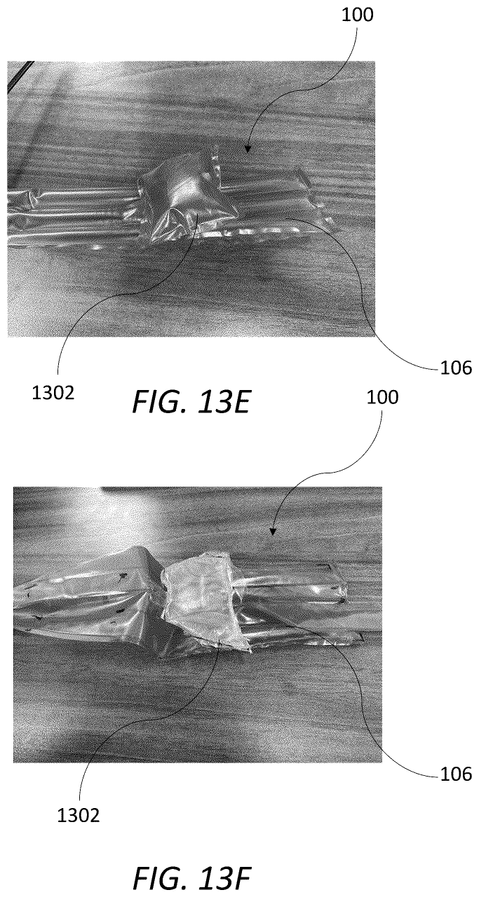

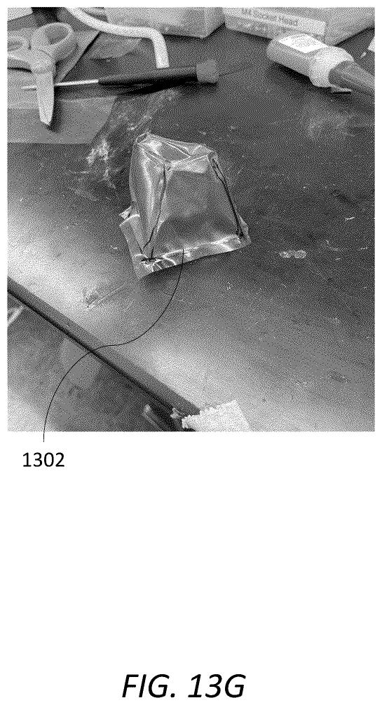

FIGS. 13A-13G depict an intubation device 100 having at least one lifting element. In some embodiments the at least one lifting element is affixed to the sagittal plane of the intubation device 100. In some embodiments, the at least one lifting element is configured to lift the soft palate and epiglottis from the pharyngeal wall to allow expansion of the primary segment 106 and secondary segment 108. In some embodiments, the at least one lifting element comprises one or more semi-rigid scales 1301 protruding from a surface of the primary segment 106, as show in FIGS. 13A-13D. In some embodiments, the semi-rigid scales 1301 may be attached to the primary segment 106 sequentially as shown in FIG. 13D. In some embodiments, the semi-rigid scales 1301 may be tethered together, as shown in FIGS. 13A-13B. In some embodiments, the semi-rigid scales 1301 scoop the surrounding tissue. In some embodiments, the semi-rigid scales 1301 burrow beneath the surrounding tissue. In some embodiments, semi-rigid scales 1301 may deploy sequentially such that leading semi-ridge scales grab the surrounding tissue before flipping and retrograding slightly. In some embodiments, the at least one lifting element comprises an inflatable member 1302 in fluid communication with the primary segments 106, as shown in FIGS. 13E-13F. In some embodiments, the inflatable member 1302 deploys and inflates at low pressure. In some embodiments, the inflatable member 1302 is any shape necessary to achieve the desired effect, namely lifting a portion of the anatomy of the patient. FIGS. 13E-13G show an inflatable member 1302 having a “pillow.” “wedge.” and “pyramidal.” shape respectively. In some embodiments, the inflatable member 1302 is configured to lift the tongue of the patient allowing passage of the primary segment to expand posteriorly of the inflatable member 1302. In some embodiments, the inflatable member 1302 may be an origami like structure comprising one or more features that deploy and inflate sequentially. In some embodiments, the inflatable member 1302 may be inverted within the primary segment 106 prior to deployment and inflation such that when pressurized the inflatable member 1302 everts to produce the desired effect.

In some embodiments, as the compliant body is pressurized, the at least one lifting element is deployed to the oropharynx just proximal to the vallecula. As pressure is supplied, the at least one lifting element inflates while extension of the primary segment 106 ceases, lifting the tissue from the pharyngeal wall and exposing a view of the hypopharynx. Once the at least one lifting element is completely inflated, the primary segment 106 continues extending through the opening created by the at least one lifting lumen.

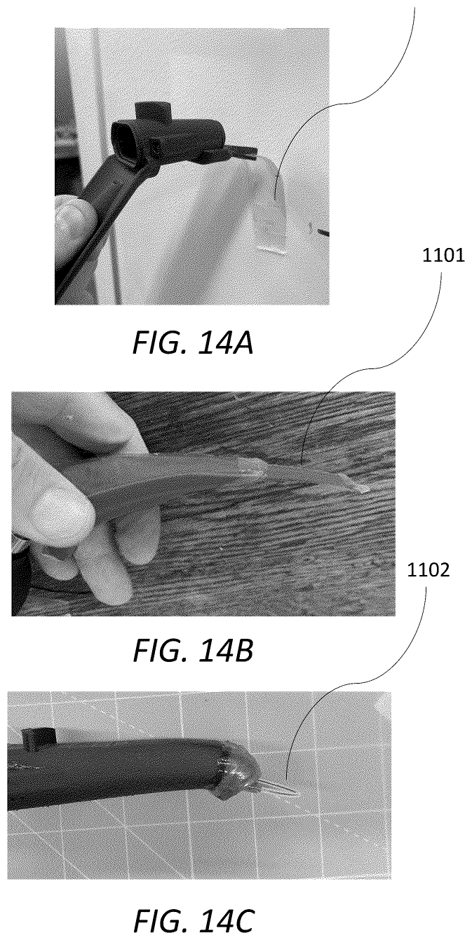

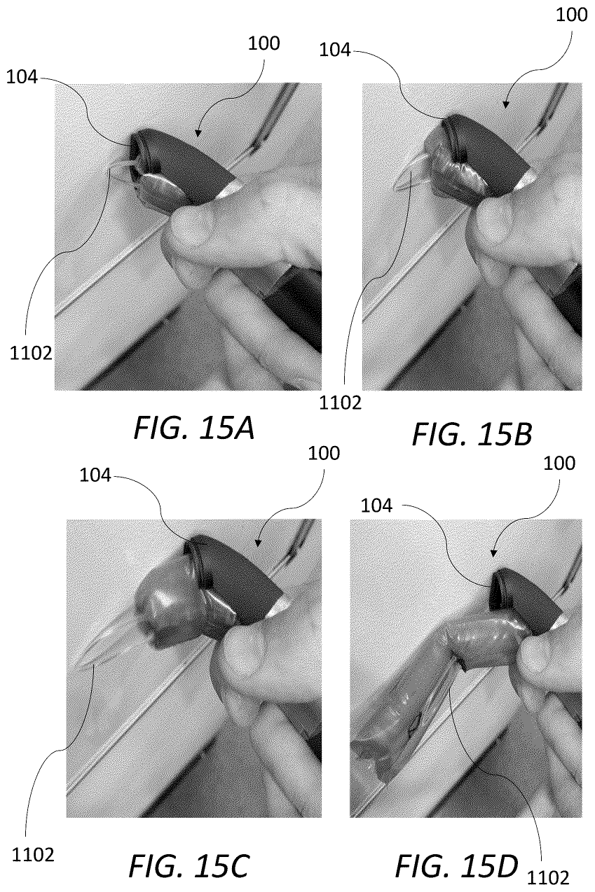

In some embodiments, the midline epiglottis defeat devices are epiglottis shimming devices. In some embodiments, the at least one shifting element comprises a flexible shim configured to slide posterior to the epiglottis upon advancement into the laryngopharynx. In some embodiments, the shim has a high aspect ratio. In some embodiments, the shim has an aspect ratio of 10:1 or higher. In some embodiments, the shim has low stiffness. In some embodiments, the shim is a low friction element allowing the shim to maneuver without encountering significant resistance by the surrounding tissue. FIGS. 14A-14C depict various embodiments of the shim. In some embodiments, the shim is a solid flexible shim 1101. In some embodiments, the shim is an encased extruded metal wire, or hollow shim 1102. In some embodiments, the hollow shim 1102 reduces friction, and allows the secondary segment 108 of the compliant body to deploy within the hollow section of the hollow shim 1102 to increase reliability across anatomies. In some embodiments, the solid flexible shim 1101 or hollow shim 1102 is attached to the compliant body, and more particularly the primary segment 106, such that when the solid flexible shim 1101 or hollow shim 1102 is advanced (i.e., extended) in response to a pressurization of the compliant body. In some embodiments, the solid flexible shim 1101 or hollow shim 1102 slips posterior to the epiglottis. Once past the epiglottis, an intubation device 100 coupled to the solid flexible shim 1101 or hollow shim 1102 is pressurized causing the compliant body to extend posterior to the solid flexible shim 1101 or hollow shim 1102, thereby passing the epiglottis without inversion (or lifting). FIGS. 15A-15D depict an embodiment of the intubation device of the present disclosure in progressive states during advancement of the compliant body posterior to the hollow shim 1102. In some embodiments, the introducer 104 datums off the pharyngeal wall such that the angle of the introducer 104 which respect to the back wall of the pharynx is fixed. Upon pressurization of the compliant body, the hollow shim 1102 advances forward. Pressurization causes the compliant body to expand posteriorly of the hollow shim 1102.

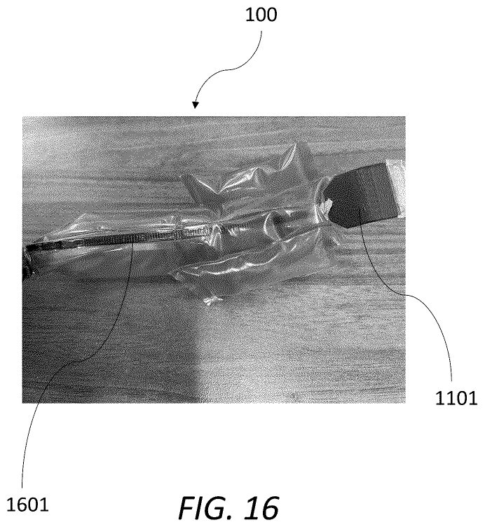

In some embodiments, the flexible shim 1101 or hollow shim 1102 is reinforced to provide rotational control of the intubation device 100. In some embodiments, the intubation device comprises a rigid vertebra traversing the length of the compliant body and coupled to the shim to provide control of the orientation of the flexible shim 1101 or hollow shim 1102 as the compliant body expands. FIG. 16 depicts an embodiment of the intubation device of the present disclosure having a rigid vertebra 1601 coupled to a flexible shim 1101.

The midline epiglottis defeat devices may further include any of the features of any embodiment of the present disclosure, for example, embodiments of the device-assisted intubation devices may be combined with embodiments of the device-assisted intubation devices and pyriform exploit epiglottis defeat devices.

### Pyriform Exploit Epiglottis Defeat Devices

The intubation devices of the present disclosure may be pyriform exploit epiglottis defeat devices. In some embodiments, the pyriform exploit epiglottis defeat devices are autonomous devices. In some embodiments, the pyriform exploit epiglottis defeat devices are asymmetric devices, such that the compliant body is configured to form, upon placement into the mouth and subsequent pressurization therein, a tertiary segment configured to extend into a first pyriform sinus (pyriform fossa) of the patient to facilitate advancement of the second segment 108 into the trachea. In some embodiments, the asymmetric pyriform exploit epiglottis defeat devices are balanced to one side of the anatomy of the patient and configured to allow the primary segment 106 and secondary segment 108 to expand medially toward the epiglottis and trachea. In some embodiments, the introducer or storage compartment 701 indicates the direction of the pyriform to allow for consistent performance of the intubation device 100.

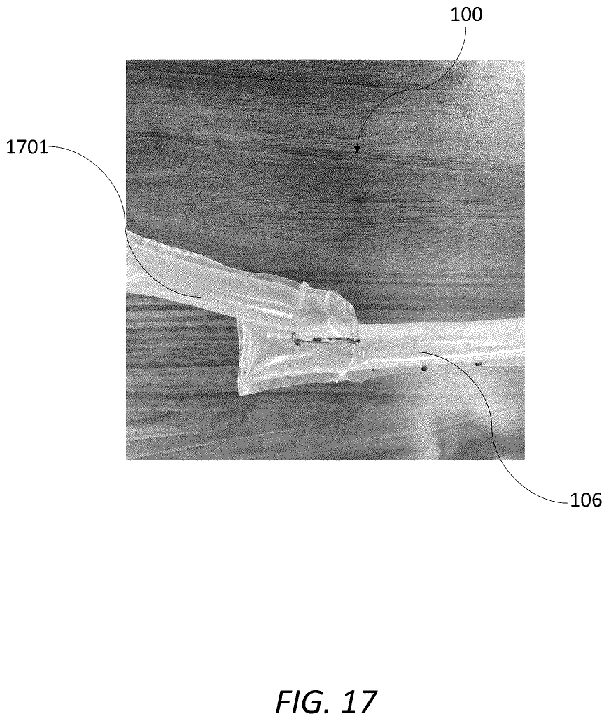

FIG. 17 depicts an asymmetric pyriform exploit epiglottis defeat devices having a tertiary segment 1701 configured to enter a first pyriform sinus of a patient. In some embodiments, the tertiary segment 1701 is configured to extend from the primary segment 106 and into the first pyriform sinus of the patient, lifting at the base of the epiglottis of the patient to facilitate advancement of the secondary segment 108 into the trachea. In some embodiments, the tertiary segment 1701 redirects the compliant body to allow the primary segment 106 to pass beneath the epiglottis. In some embodiments, the tertiary segment 1701 is configured to extend from the primary segment 106 into the first pyriform sinus of the patient, and wherein the secondary segment 108 is configured to extend from the primary segment 106 into the trachea of the patient.

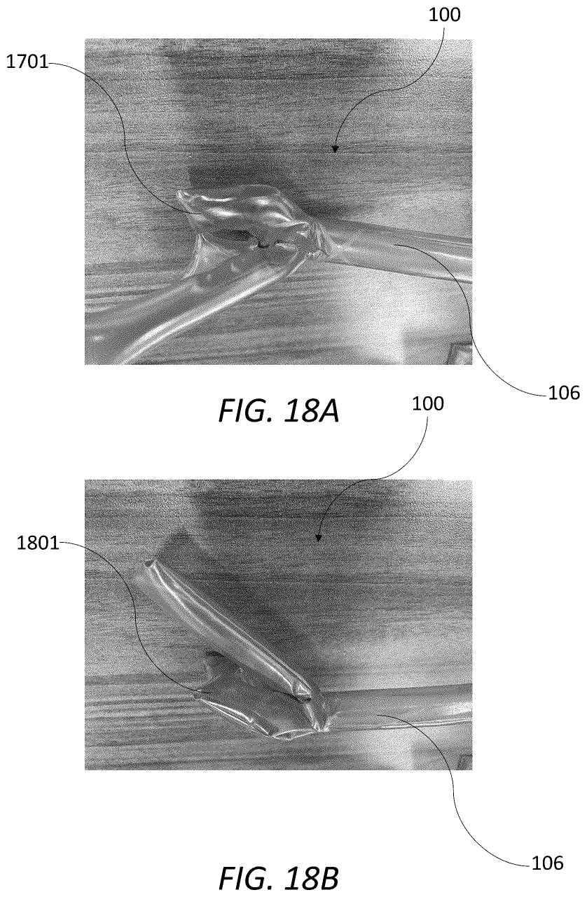

In some embodiments, the pyriform exploit epiglottis defeat devices are symmetric exploit devices, such that the compliant body is configured to form, upon placement into the mouth and subsequent pressurization therein, configured to form, upon placement into the mouth and subsequent pressurization therein, a tertiary segment configured to extend from the primary segment 106 into a first pyriform sinus (pyriform fossa) of the patient and a quaternary segment configured to extend from the primary segment 106 and into a second pyriform sinus of the patient. FIGS. 18A-18B depict a symmetric pyriform exploit epiglottis defeat devices having a tertiary segment 1701 configured to enter a first pyriform sinus of a patient and a quaternary segment 1801 configured to enter into a second pyriform sinus of the patient. In some embodiments, the tertiary segment 1701 is configured to extend from the primary segment 106. In some embodiments, the quaternary segment 1801 is configured to extend from the primary segment 106. In some embodiments, the tertiary segment 1701 and quaternary segment 1801 lift at the base of the epiglottis of the patient to facilitate advancement of the secondary segment 108 into the trachea.

In some embodiments, the cross-sectional area of the tertiary segment 1701 and quaternary segment 1801 is less than the cross-sectional area of the primary segment 106. Generally, the diameter and length of both the tertiary segment 1701 and quaternary segment 1801 can be predetermined according to physiology type or measured physical characteristics of a patient being intubated. e.g., age, gender, according to physical measurements of throat structure, etc. In practice, a practitioner can select an intubation device 100 appropriately sized based upon the physiology of the patient.

The pyriform exploit epiglottis defeat devices may further include any of the features of any embodiment of the present disclosure, for example, embodiments of the pyriform exploit epiglottis defeat devices may be combined with embodiments of the midline epiglottis defeat devices and device-assisted intubation devices.

### Methods of Use

In some embodiments, the present disclosure provides a method for intubating a patient placing a compliant body into a bodily orifice of the patient (as shown in FIG. 1A, for example). In some embodiments, the method further comprises the step of pressurizing the compliant body through at least one patency thereof so that a primary segment 106 of the compliant body everts and extends into the back of a laryngopharynx of the patient (as shown in FIG. 1B, for example) and a secondary segment 108 of the compliant body thereafter everts and extends from the primary segment 106 and into a trachea of the patient (as shown in FIG. 1C, for example). In some embodiments, the method further comprises the step of providing ventilation from the bodily orifice to the trachea via the pressurized compliant body.

In some embodiments, the at least one patency comprises a first patency 201 and a separate second patency 202, wherein the compliant body comprises one or more walls 203 defining the first and second patency 202, wherein the pressurization via the first patency 201 defines the primary segment 106 and secondary segment 108, and the second patency 202 provides the ventilation. In some embodiments, the second patency 202 directly provides ventilation for the patient therethrough.

In some embodiments, the step of providing ventilation via the separate second patency 202 comprises advancing an endotracheal tube through the second patency 202 and providing ventilation through the endotracheal tube. In some embodiments, the first and second patency 202 are configured to be pressurized by different pressurization streams.

In some embodiments, the method further comprises maintaining patency of the second segment 108 with a cuff coupled to a distal portion of the secondary segment 108. In some embodiments, the cuff is at least partially rigid. In some embodiments, the cuff comprising expanding the cuff. In some embodiments, the expanding the cuff comprises inflating the cuff. In some embodiments, the cuff is inflated with a pressure source independent than another pressure source for the primary segment 106 and secondary segment 108.

In some embodiments, the method further comprises the step of orienting the primary segment 106 to a preferred orientation to the trachea as the primary segment 106 is pressurized. In some embodiments, orienting the primary segment 106 comprises pressurizing a lateral sub-segment 802 of the primary segment 106 extending laterally from a first sub-segment 801.

In some embodiments, the method further comprises the step of shifting the epiglottis to facilitate extension of the secondary segment 108 into the trachea. In some embodiments, the step of shifting the epiglottis comprises laterally extending at least one lifting element from the primary segment 106 as the primary segment 106 is being pressurized. In some embodiments, the step of shifting the epiglottis comprises inflating at least one lifting element as the primary segment 106 is being pressurized. In some embodiments, the step of shifting the epiglottis comprises extending a flexible shim 1101 from the primary segment 106 in response to pressurization thereof so that the flexible shim 1101 slides posteriorly of the epiglottis.

In some embodiments, the step of pressurizing the compliant body through the at least one patency thereof extends a tertiary segment 1701 of the compliant body into at least one pyriform sinus of the patient. In some embodiments, the tertiary segment 1701 extends from the primary segment 106 and into the first pyriform sinus of the patient, lifting an epiglottis of the patient to facilitate advancement of the secondary segment 108 into the trachea. In some embodiments, the tertiary segment 1701 extends from the primary segment 106 into the first pyriform sinus of the patient, and wherein the secondary segment 108 extends from the primary segment 106 into the trachea of the patient.

In some embodiments, the compliant body forms, upon placement into the bodily orifice and subsequent pressurization therein, a quaternary segment 1801 extending into a second pyriform sinus of the patient. In some embodiments, the quaternary segment 1801 extends from the primary segment 106 and into the second pyriform sinus lifting an epiglottis of the patient to facilitate advancement of the secondary segment 108 into the trachea.

In some embodiments, the at least one patency directly provides ventilation for the patient therethrough.

In some embodiments, the step of providing ventilation via the at least one patency comprises advancing an endotracheal tube through the at least one patency and providing ventilation through the endotracheal tube.

In some embodiments, the method further comprises retracting the compliant body from the trachea and the bodily orifice. In some embodiments, retracting the compliant body comprises applying negative pressure to the compliant body.

In some embodiments, the bodily orifice is a mouth of the patient. In some embodiments, the bodily orifice is a nostril of the patient.

Although the above steps describe methods of intubating a patient in accordance with many embodiments, a person of ordinary skill in the art will recognize many variations based on the teaching described herein. The steps may be completed in a different order. Steps may be added or omitted. Some of the steps may comprise sub-steps. Many of the steps may be repeated as often as beneficial or advantageous.

While preferred embodiments of the present disclosure have been shown and described herein, it will be obvious to those skilled in the art that such embodiments are provided by way of example only. Numerous variations, changes, and substitutions will be apparent to those skilled in the art without departing from the scope of the present disclosure. It should be understood that various alternatives to the embodiments of the present disclosure described herein may be employed without departing from the scope of the present invention. Therefore, the scope of the present invention shall be defined solely by the scope of the appended claims and the equivalents thereof.

What is claimed is:

1. A device for intubation of a subject, the device comprising:
a compliant body configured to form, upon placement into a bodily orifice of a subject and subsequent pressurization therein, (i) a primary segment configured to extend to the back of the laryngopharynx of the subject, (ii) a secondary segment configured to extend from the first segment and into a trachea of the subject, and (iii) at least one patency from the first segment to the second segment to provide a pathway from a bodily orifice of the subject to the trachea of the subject.

2. The device of claim 1, wherein the compliant body comprises one or more walls defining a first patency and a second patency, the first patency permitting pressurization of the compliant body to form the primary and secondary segments, and the second patency extending from the primary segment into the secondary segment to provide the pathway from the bodily orifice of the subject into the trachea of the subject.

3. The device of claim 2, wherein the first and second patency are configured to be pressurized by different pressurization streams.

4. The device of claim 2, further comprising an introducer coupled to the compliant body and configured for placement at least partially into the bodily orifice of the subject, wherein the introducer comprises one or more of a first access port or a second access port, the first access port being open to the first patency and the second access port being open to the second patency.

5. (canceled)

6. The device of claim 2, wherein the second patency is configured to provide ventilation for the subject directly through the pathway provided therefrom.

7. The device of claim 2, wherein the portion of the one or more walls defining second patency is supported by one or more rigid elements.

8-10. (canceled)

11. The device of claim 2, wherein the second patency is disposed at least partially within the first patency.

12. The device of claim 2, wherein the first and second patency are at least partially coaxial.

13. The device of claim 1, wherein the device further comprises a cuff coupled to the secondary segment and configured to protect the at least one patency.

14. (canceled)

15. The device of claim 13, wherein the cuff is expandable.

16-17. (canceled)

18. The device of claim 13, wherein the cuff is coupled to a distal portion of the secondary segment.

19. The device of claim 1, further comprising an introducer coupled to the compliant body and configured for placement and securement at least partially into the bodily orifice of the subject.

20-25. (canceled)

26. The device of claim 1, further comprising an introducer coupled to the compliant body, the introducer being configured for placement and securement at least partially into the bodily orifice of the subject.

27. (canceled)

28. The device of claim 1, wherein the primary segment comprises a first sub-segment and one or more lateral sub-segments, the one or more lateral sub-segments being partially closed to fluid communication with the primary sub-segment.

29. The device of claim 1, wherein the primary segment has a non-circular cross-section such that the primary segment orients to a preferred orientation to the trachea as the primary segment is pressurized.

30-31. (canceled)

32. The device of claim 1, further comprising at least one shifting element configured to shift the surrounding tissue of the subject and facilitate advancement of the secondary segment into the trachea.

33. The device of claim 1, further comprising at least one shifting element configured to shift an epiglottis of the subject and facilitate advancement of the secondary segment into the trachea.

34-37. (canceled)

38. The device of claim 33, wherein the at least one shifting element comprises a flexible shim configured to slide posteriorly of the epiglottis upon advancement into the laryngopharynx.

39-44. (canceled)

45. The device of claim 1, wherein the bodily orifice is a mouth of the subject.

46. (canceled)

47. The device of claim 1, wherein one or more of the primary or secondary segments are configured to evert in response to the pressurization.

48. The device of claim 1, wherein the secondary segment has a smaller cross-sectional area than the primary segment.

49. The device of claim 1, wherein the primary segment has a predetermined shape configured to place and orient the secondary segment toward the trachea.

50. The device of claim 1, wherein the primary and secondary segments are unitarily formed.

51-52. (canceled)

53. The device of claim 1, wherein at least a portion of the compliant body is supported by one or more rigid elements.

54.-94. (canceled)
